(19) United States
(12) Patent Application Publication
(10) Pub. No.: US 2022/0143425 A1
(43) Pub. Date: May 12, 2022

(54) METHOD AND APPARATUS FOR TAKING INTO ACCOUNT SUSCEPTIBILITY DEVIATIONS IN MR-BASED THERAPY PLANNING

(71) Applicant: Siemens Healthcare GmbH, Erlangen (DE)

(72) Inventors: Manuel Schneider, Erlangen (DE); Martin Requardt, Nürnberg (DE); Himanshu Bhat, Newton, MA (US); Matthias Drobnitzky, Spardorf (DE); Xin Miao, Medford, MA (US); Xinyuan Cui, Los Angeles, CA (US)

(21) Appl. No.: 17/520,974

(22) Filed: Nov. 8, 2021

(30) Foreign Application Priority Data
Nov. 12, 2020 (DE) 10 2020 214 255.6

(51) Int. Cl. A61N 5/10; G01R 33/48; G01R 33/44; G01R 33/565; G01R 33/56; G06T 11/00

(52) U.S. Cl. CPC A61N 5/1039; G01R 33/4828; G01R 33/443; G06T 2210/41; G01R 33/5608; G06T 11/003; G01R 33/56563

(57) ABSTRACT

Systems and methods for taking into account susceptibility deviations in magnetic-resonance-based therapy planning by a magnetic resonance tomography unit. A B0 field map is determined by the magnetic resonance tomography unit. A location blur distribution is determined from the B0 field map and from the location blur distribution in turn, a parameter of an image acquisition as a function of the location blur distribution, in such a way that an image acquisition brings about a reduced location blur with the determined parameter.

### CROSS REFERENCE TO RELATED APPLICATIONS

This patent document also claims the benefit of DE 10 2020 214 255.6 filed on Nov. 12, 2020, which is hereby incorporated in its entirety by reference.

### FIELD

Embodiments relate to a method for taking into account susceptibility deviations in MR-based therapy planning by a magnetic resonance tomography unit.

### BACKGROUND

Magnetic resonance tomography units are imaging apparatuses that align nuclear spins of the examination object with a strong external magnetic field in order to image an examination object and excite the nuclear spins to precession around the alignment by an alternating magnetic field. The precession or return of the spins from the excited state to a lower energy state in turn generates an alternating magnetic field received via antennae in response.

With the aid of magnetic gradient fields, a location coding is impressed on the signals that subsequently provides an assignment of the received signal to a volume element. The received signal is then evaluated, and a three-dimensional imaging representation of the examination object provided. To receive the signal, local receiving antennae, e.g., local coils, may be used, that are arranged directly on the examination object to achieve a better signal-to-noise ratio. The receiving antennae may also be installed in a patient couch.

Therapy planning is often carried out for radiation treatments, needle biopsies, ablation by highly focused ultrasonic waves or other interventions, in which the exact location of an organ or a tumor must be known in order either to carry out the intervention at the correct location and/or to avoid damage to the surrounding area.

The magnetic resonance tomography unit provides good imaging of organs as it primarily detects the protons and in so doing may also distinguish different chemical bonding environments. However, the exact detection of the location depends on the magnetic field and thus its homogeneity. Even if the magnetic field is almost ideal as a result of construction and/or is homogenized by active or passive shimming, a patient again introduces inhomogeneities due to different susceptibility of different tissues, that cannot be predicted sufficiently accurately and may sometimes only be partially remedied by shim coils. For these reasons, therapy planning may be performed with a computed tomography unit as the high-energy X-rays are insensitive to deflection due to fluctuations in susceptibility or slight fluctuations in density in the body.

### BRIEF SUMMARY AND DESCRIPTION

The scope of the present invention is defined solely by the appended claims and is not affected to any degree by the statements within this summary. The present embodiments may obviate one or more of the drawbacks or limitations in the related art.

Embodiments improve therapy planning using a magnetic resonance tomography unit.

The method relates to taking into account susceptibility deviations in magnetic-resonance-based therapy planning by a magnetic resonance tomography unit. Therapy planning refers to the preparation of a therapy, treatment or also an examination in which precise information about the location or relative position of an organ or of a part of an organ is required. This may be the case, for example, if the organ or a part thereof is to be subjected to an intervention such as a needle biopsy or is to be subjected to a targeted local treatment from the outside for which the precise geometric location must be known, such as local irradiation with particles, X-rays, or gamma rays or also ultrasound. Conversely, it may also be important that a sensitive organ is to be protected from side effects of the therapy or that shadowing by organs has to be taken into account in therapy planning.

The method includes the step of acquiring a B0 field map by the magnetic resonance tomography unit. In this case, a B0 field map is understood to mean an item of information that, at a plurality of locations, for example within the Field of View (FoV) of the magnetic resonance tomography unit, respectively assigns a variable that is a measure of the static magnetic field B0 of the magnetic resonance tomography unit prevailing there. This variable may be, for example, the size of the magnetic field in Tesla, a relative size indication with regard to the local field relative to a predetermined value for B0, or also a variable dependent on the magnetic field, such as the Larmor frequency of a nuclear spin. The B0 field map may be 3-dimensional, but also 2-dimensional. For example, B0 deviations may be determined from a phase difference of different recorded gradient echoes.

In a further step, a location blur distribution is determined from the B0 field map. This may be done, for example, by the controller of the magnetic resonance tomography unit, or by a dedicated computing unit. A relation is referred to as a location blur distribution that assigns a value to a plurality of locations. The value is a measure of a deviation of the location assigned to a measured value in a measurement by the magnetic resonance tomography unit relative to the geometric location. For example, the true location of an object or voxel in magnetic resonance tomography may deviate from the location indicated by the pixel by which the object or voxel is represented in the magnetic resonance image. The location blur function may be in the form of a table of values, a matrix, or a function, for example. In the simplest case, the location blur is proportional to the deviation of the respective magnetic field value from a predetermined value B0.

In a further step, a parameter of an image acquisition is determined as a function of the location blur distribution such that an image acquisition with the parameter leads to reduced location blur in a predetermined region. For example, the parameter may be changed in such a way that the location blur for a predetermined voxel is reduced, or an average local deviation is minimized. One possibility is to increase the readout bandwidth. The predetermined region may relate, for example, to an organ to be treated or, conversely, to a sensitive organ that is to be protected from undesired damage during the subsequent therapy or treatment. It may be regarded as location blur if the location blur in the predetermined region falls below a predetermined limit value. For example, the limit value may specify a maximum location blur in a predetermined direction or for the amount of location blur, so that the location blur is, for example, less than 10 mm, 5 mm, 2 mm, or 1 mm.

The method provides location blur to be taken into account when planning a therapy.

The magnetic resonance tomography unit includes similar advantages as the method.

In an embodiment, the method further includes a step of carrying out image acquisition with the determined parameter from the magnetic resonance tomography unit. The image acquisition may be carried out using the same magnetic resonance tomography unit as the acquisition of the B0 field map.

The image acquisition may take place with a second magnetic resonance tomography unit, for example, if the parameter of the image acquisition relates to the B0 magnetic field strength. This is because a possibility for reducing the blur caused by the patient and their susceptibility discontinuities is image acquisition with a maximum B0 field determined from the location blur. The parameter value may be output from the magnetic resonance tomography unit on an output device, so that an operator performs the image acquisition with another magnetic resonance tomography unit with this magnetic field strength. The magnetic resonance tomography unit may also be configured to change its magnetic field to the value.

Subsequently, an image is reconstructed from the data acquired in the image acquisition and displayed to a user on an output device.

An image with a required low location blur may be provided.

In an embodiment of the method, the step of acquiring a B0-field map includes a Dixon gradient echo sequence.

Dixon gradient echo sequences with a high bandwidth may be detected, that leads to lower location errors of a location blur distribution determined therefrom.

In an embodiment of the method, a set of parameters is predetermined for the image acquisition. For example, starting values for the parameters for the sequences used may be specified from a table. An iterative optimization method for improving the parameters may be provided. In the step of determining the location blur distribution, the determination is carried out as a function of a parameter of the parameter set. The determined location blur distribution may depend on several or all of the parameters.

The inclusion of the parameter provides a realistic image of the location blurring of the image to be acquired later and also allows improvements or degradations caused by parameter changes to be recognized.

In an embodiment of the method, the determination of the location blur distribution includes a weighted location deviation. For example, a histogram may be created that weights a location deviation with the number of pixels affected thereby. This may also take place, for example, only for a predetermined region, for example, a region of interest (RoI).

The weighted distribution provides a quick and easy assessment of whether the distortion is, for example, a large-scale or punctiform distortion.

In an embodiment of the method, the magnetic resonance tomography unit includes an image acquisition region, for example a “field of view” (FoV). The location blur distribution is determined only for a genuine subset of the image acquisition region. This may, for example, only include one volume in which a particularly sensitive organ is arranged or a tissue to be examined or treated.

By limiting the volume in which the location blur is relevant and must be recorded, the determination of the B0 field map and location blur distribution may be accelerated.

In an embodiment of the method, in the step of setting the parameter of the image acquisition, the parameter is set in such a way that a location blur in a predetermined subset of the image acquisition region is reduced. Parameters and their setting are explained in more detail hereinafter.

The location blur may be reduced in an advantageous manner by adapting the parameter(s), and the therapy may thus be made better and safer.

In an embodiment of the method, the parameter to be set is one from the group of readout bandwidth, readout direction, type of sequence and/or trajectory. Several parameters of this group may be changed at the same time or the parameter and one or more parameters not from the group.

For example, an increased readout bandwidth leads to a lower sensitivity for B0 fluctuations and thus to lower location blur, but with a reduction in the SNR. Sequences such as MARS (metal artefact reduction sequence), e.g., WARP, VAT or SEMAC, also reduce the location blur. Radial or spiral trajectories reduce the displacement of the pixels in one direction compared to undirected blur. Phase coding in three spatial directions reduces or eliminates the location blur at the expense of extended acquisition times.

The different parameters make the reduction of the location blur flexible and thus allow an optimum result to be achieved, even in different situations, with regard to the patient, location, and position of the region to be examined, type of magnetic resonance tomography unit, SAR limit values and available time.

In an embodiment of the method, the method furthermore includes the step of executing the image reconstruction as a function of the location blur distribution. An image reconstruction method may be selected that, with the data already acquired, reduces the location blur for the resulting pixels, at least in a predetermined subset. Examples of such reconstruction methods may be found in the publication N. L. Dispenza, Y. M. Rodriguez, R. T. Constable, et al. “Imaging Beyond the Homogeneous Radius in Clinical Magnets”. In: Proceedings of the 27th Annual Meeting of ISMRM, Montreal, QC, Canada, #4517, 2019.

The selection of a reconstruction method as a function of the location blur distribution also makes it possible to adapt the achieved location blur for the pixels to the requirements of the examination even after the recording of the measured values.

In an embodiment of the method, the method furthermore includes the step of performing image acquisition with a computed tomography unit as a function of the location blur distribution. By the determined location blur distribution and the possible changes in the image acquisition parameters and reconstruction methods, the result will be that sufficient location accuracy for therapy planning cannot be achieved with the magnetic resonance tomography unit and that instead conventional computed tomography is to be carried out.

As a result, the outlay for an unnecessary magnetic resonance recording, as it is not targeted, may be saved and the process for the patient may be simplified.

### BRIEF DESCRIPTION OF THE FIGURES

FIG. 1 depicts a diagrammatic view of a magnetic resonance tomography unit for performing the method according to an embodiment.

FIG. 2 depicts a diagrammatic flow chart of the method according to an embodiment.

### DETAILED DESCRIPTION

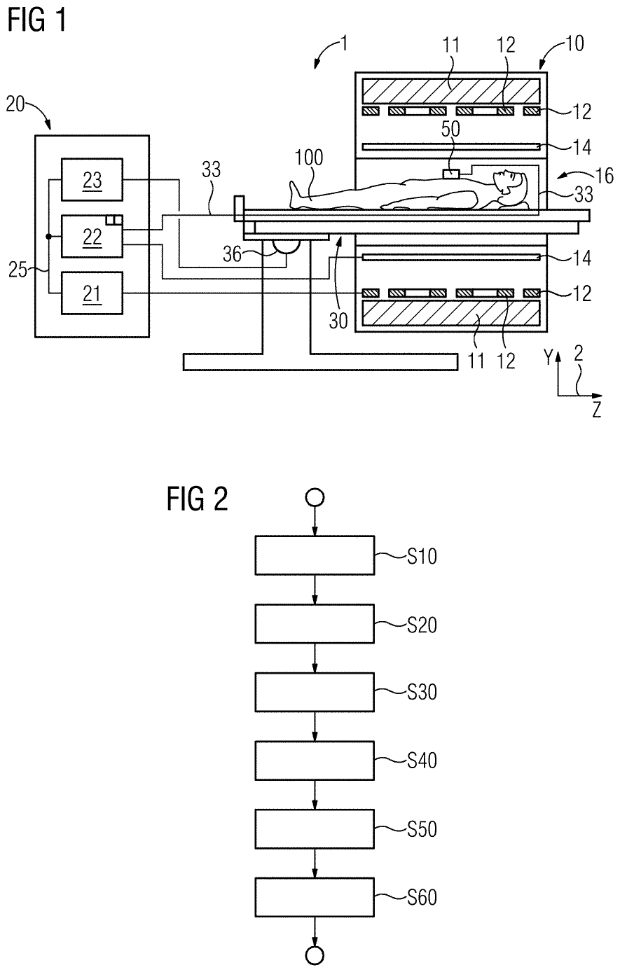

FIG. 1 depicts a diagrammatic view of an embodiment of a magnetic resonance tomography unit 1 for carrying out the method according to an embodiment.

The magnet unit 10 includes a field magnet 11 that generates a static magnetic field B0 for aligning nuclear spins of samples or of the patient 100 in a receiving area. The receiving area is characterized by an extremely homogenous static magnetic field B0, the homogeneity relating to the magnetic field strength or the magnitude. The receiving area is almost spherical and arranged in a patient tunnel 16 that extends in a longitudinal direction 2 through the magnet unit 10. A patient couch 30 may be moved in the patient tunnel 16 by the positioning unit 36. The field magnet 11 may be a superconducting magnet that may provide magnetic fields with a magnetic flux density of up to 3T, and in the case of the latest devices even higher. However, permanent magnets or electromagnets with normally conducting coils may also be used for lower field strengths.

Furthermore, the magnet unit 10 includes gradient coils 12 that are configured to superimpose variable magnetic fields on the magnetic field B0 in three spatial directions to spatially differentiate the acquired imaging regions in the examination volume. The gradient coils 12 may be coils of normally conducting wires that may generate fields in the examination volume that are orthogonal to one another.

The magnet unit 10 includes a body coil 14 that is configured to radiate a radio frequency signal supplied via a signal line into the examination volume and to receive resonance signals emitted by the patient 100 and to output them via a signal line. Hereinafter, the term transmitting antenna denotes an antenna via which the radio frequency signal for exciting the nuclear spins is emitted. This may be the body coil 14, but also a local coil 50 with a transmission function.

A control unit 20 supplies the magnet unit 10 with the various signals for the gradient coils 12 and the body coil 14 and evaluates the received signals.

The control unit 20 includes a gradient control 21 that is configured to supply the gradient coils 12 via supply lines with variable currents that provide the desired gradient fields in the examination volume in a time-coordinated manner.

The control unit 20 includes a radio frequency unit 22 that is configured to generate a radio frequency pulse with a predetermined time profile, amplitude, and spectral power distribution for excitation of a magnetic resonance of the nuclear spins in the patient 100. Pulse outputs in the range of kilowatts may be achieved. The excitation signals may be emitted into the patient 100 via the body coil 14 or also via a local transmitting antenna.

A controller 23 communicates via a signal bus 25 with the gradient controller 21 and the radio frequency unit 22.

A local coil 50 is arranged on the patient 100 and is connected to the radio frequency unit 22 and the receiver thereof via a connecting line 33. The body coil 14 may be a receiving antenna.

FIG. 2 depicts an embodiment of the method.

In a step S10, the magnetic resonance tomography unit detects a B0 field map with the control unit 20, the field magnet 10, the gradient coils 12, the transmitting antenna, and the receiving antenna. For example, information relating to B0 deviations may be determined by single or dual gradient echo sequences. B0 field deviations may be determined from the phase of interleaved dual-echo EPI sequences. In an embodiment, a Dixon gradient echo sequence is used.

In order to record the B0 field map, a predetermined set of parameters is used that is determined by the sequence used or, conversely, defines it. Parameters may be, for example, frequency, spectral distribution and amplitude of excitation pulses, strength, and direction of gradient fields as well as their temporal course and temporal arrangement with respect to one another, and the B0 field strength of the field magnet 11.

In a further step S20, the control unit 20 determines a location blur distribution from the B0 field map. The control unit may have its own computing unit or use a computing unit for image reconstruction or also use the controller 23 for image acquisition. In the simplest case, it may be assumed that the location blur is proportional to a deviation in the B0 field strength. However, the location blur may also include a directional dependence that differs in a direction perpendicular to a boundary between two regions of different susceptibility from the direction parallel to the interface.

The determined location blur may depend on the predetermined set of parameters in the acquisition of the B0 field map, as will be explained hereinafter with regard to the selection of the parameter for image acquisition.

For a simpler assessment of adequate image quality with regard to geometric distortions, the location blur distribution may be reduced to a smaller amount of data. For example, relevant image regions and irrelevant image regions may be weighted differently in the assessment. Regions that include, for example, the tissue to be examined or treated or, conversely, sensitive organs that must not be damaged, may be regarded as relevant image regions. The data may thus also be reduced, for example, to a single measure that is formed by a sum of the location blur amounts multiplied by the weighting factor for all voxels. Histograms with an optionally weighted number of voxels in a predetermined location blur region may be used.

The location blur distribution may be determined only for a subset of the voxels, for example, in relevant image regions. Conversely, the location blur for voxels in irrelevant regions could also be given a correspondingly low weighting or multiplied by a weighting factor of zero.

In a step S30, the control unit 20 determines a parameter of an image acquisition from the location blur distribution, or as a function of the location blur distribution. As already explained above with regard to step S20, the location blur may depend on the direction relative to a susceptibility limit. Thus, for example, by selecting the readout direction as a parameter of the image acquisition relative to the susceptibility limit, the location blur may be reduced.

Further parameters of the image acquisition with which the location blur may be influenced in the case of image acquisition for given susceptibility discontinuities are, for example, also readout bandwidth, type of sequence, and/or trajectory.

However, the location blur in the case of a predetermined susceptibility jump also depends on the absolute magnetic field strength used. In order to limit the location blur to a required maximum, the control unit 20 may, for example, determine a maximum magnetic field strength B0 for which this is fulfilled as a parameter. This value may be output to an operator on an output unit. The operator may then, for example, execute an image acquisition of the patient 100 with another magnetic resonance system of suitable field strength B0 in a step S40.

In the case of extreme susceptibility jumps, the parameter may also indicate in a broader sense that the imaging cannot be carried out with an available magnetic resonance tomography unit 1 with sufficient accuracy and therefore the image acquisition must take place on a different modality, for example a computed tomography unit. An instruction as a corresponding parameter is then output by the magnetic resonance tomography unit 1 to the operator.

The same magnetic resonance tomography unit 1 may carry out an image acquisition with the determined parameter in step S40. This may, for example, be an image acquisition with a changed readout direction. In suitable magnetic resonance systems 1 with conventional field magnets 11 and broadband reception technology, however, it may be possible to reduce the magnetic field strength B0 to a maximum value determined as a parameter.

In a step S50, the magnetic resonance tomography unit 1 performs an image reconstruction with the acquired magnetic resonance signals. In the image reconstruction, the parameters may be modified in the direction of a lower location deviation, or a suitable image reconstruction algorithm may be selected as a function of the parameter.

In step S60, the control unit 20 outputs the pictorial representation on an output device, for example, a display.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for taking into account susceptibility deviations in magnetic-resonance-based therapy planning by a magnetic resonance tomography unit, the method comprising:
acquiring a B0 field map by the magnetic resonance tomography unit;
determining a location blur distribution from the B0 field map; and
determining at least one parameter of an image acquisition as a function of the location blur distribution in such a way that an image acquisition with this determined parameter brings about a reduced location blur.

2. The method of claim 1, further comprising:
executing an image acquisition with the determined parameter;
executing an image reconstruction with data of the image acquisition; and
outputting the reconstructed image on an output device.

3. The method of claim 1, wherein acquiring the B0 field map comprises a Dixon gradient echo sequence.

4. The method of claim 1, wherein a parameter set is predetermined for the image acquisition, and for determining the location blur distribution, the location blur distribution is carried out as a function of a parameter of the parameter set.

5. The method of claim 1, wherein determining the location blur distribution comprises a weighted location deviation.

6. The method of claim 1, wherein the magnetic resonance tomography unit includes an image acquisition region, and the determination of the location blur distribution only takes place for a genuine subset of the image acquisition region.

7. The method of claim 6, wherein for determining at least one parameter of the image acquisition, the at least one parameter is set in such a way that a location blur is reduced in a predetermined subset of the image acquisition region.

8. The method of claim 1, wherein the at least one parameter is at least one of readout bandwidth, readout direction, type of sequence, or trajectory.

9. The method of claim 1, further comprising:
executing the image reconstruction as a function of the location blur distribution.

10. The method of claim 1, further comprising:
performing an image acquisition with a computed tomography unit as a function of the location blur distribution.

11. A magnetic resonance tomography unit comprising:
a control unit;
a field magnet;
one or more gradient coils;
a transmitting antenna; and
a receiving antenna;
wherein the magnetic resonance tomography unit is configured to:
acquire a B0 field map with the control unit, the field magnet, the one or more gradient coils, the transmitting antenna, and the receiving antenna;
determine a location blur distribution with the control unit from the B0 field map; and
determine, with the control unit, at least one parameter of an image acquisition as a function of the location blur distribution which leads to a reduction in the location blur.

12. The magnetic resonance tomography unit of claim 11, wherein the magnetic resonance tomography unit is further configured to:
perform an image acquisition using the determined parameter with the control unit, the field magnet, the one or more gradient coils, the transmitting antenna, and the receiving antenna;
perform an image reconstruction with the control unit using data from the image acquisition; and
output the reconstructed image on an output device.

13. The magnetic resonance tomography unit of claim 11, wherein acquiring the B0 field map comprises a Dixon gradient echo sequence.

14. The magnetic resonance tomography unit of claim 11, wherein a parameter set is predetermined for the image acquisition, and for determining the location blur distribution, the location blur distribution is carried out as a function of a parameter of the parameter set.

15. The magnetic resonance tomography unit of claim 11, wherein determining the location blur distribution comprises a weighted location deviation.

16. The magnetic resonance tomography unit of claim 11, wherein the magnetic resonance tomography unit includes an image acquisition region, and the determination of the location blur distribution only takes place for a genuine subset of the image acquisition region.

17. The magnetic resonance tomography unit of claim 16, wherein for determining at least one parameter of the image acquisition, the at least one parameter is set in such a way that a location blur is reduced in a predetermined subset of the image acquisition region.

18. A non-transitory computer implemented storage medium that stores machine-readable instructions executable by at least one processor, the machine-readable instructions comprising:
acquiring a B0 field map by a magnetic resonance tomography unit;
determining a location blur distribution from the B0 field map; and
determining at least one parameter of an image acquisition as a function of the location blur distribution in such a way that an image acquisition with this determined parameter brings about a reduced location blur.

19. The non-transitory computer implemented storage medium of claim 18, wherein the machine-readable instructions further comprise:
executing an image acquisition with the determined parameter;
executing an image reconstruction with data of the image acquisition; and
outputting the reconstructed image on an output device.

20. The non-transitory computer implemented storage medium of claim 18, wherein acquiring the B0 field map comprises a Dixon gradient echo sequence.
